(19) United States
(12) Patent Application Publication
(10) Pub. No.: US 2002/0173831 A1
(43) Pub. Date: Nov. 21, 2002

(54) Acupuncture needle

(76) Inventor: Paulo de Tarso Costa Dos Santos, Munchen (DE)

Correspondence Address: BEYER WEAVER & THOMAS LLP, P.O. BOX 778, BERKELEY, CA, 94704-0778, US

(21) Appl. No.: 10/019,597

(22) PCT Filed: Apr. 25, 2001
(86) PCT No.: PCT/DE01/01558

(30) Foreign Application Priority Data
Apr. 25, 2000 (DE) 200 07 456.3

(51) Int. Cl. A61N 5/067

(52) U.S. Cl. 607/88; 606/189

(57) ABSTRACT

Acupuncture needle having a grasping and a puncturing end comprising a hollow needle body and a coaxially located light conducting element therein, a reception and emission surface for biophoton radiation at the distal puncturing end of the needle and a prism with a 180° reflection and in light conducting contact with the light conducting element at the proximal grasping end of the needle.

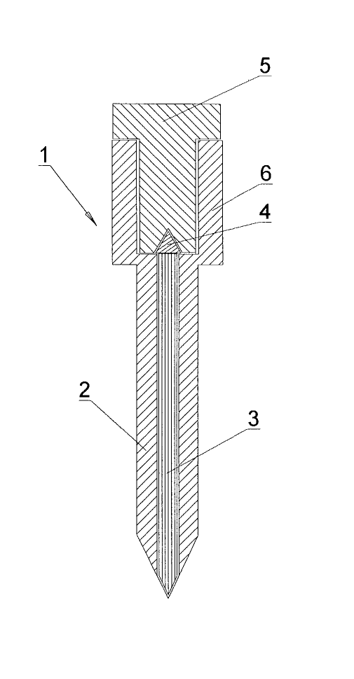

The present invention relates to acupuncture needles. According to Chinese Medicine and recent western knowledge, acupuncture needles are placed in skin areas characterised by a particular electrical conductivity. According to findings in biophoton research these skin areas show a considerably higher biophoton emission than other skin areas. While known acupuncture needles are perfectly capable to take advantage of the electrical conductivity they are neutral or unsusceptible with respect to biophoton radiation.

U.S. Pat. No. 5,250,068 shows an acupuncture needle in which a light conduction material is comprised in a needle body in order to transmit light and/or warmth from an outer source to the puncture location of a body. This shall produce a healing effect on the body area located at the puncture point through energy coming from outside the body.

The object of the present invention is to realise an acupuncture needle capable to exploit biophoton radiation from inside the body and to restitute it to the body without exiting the acupuncture needle.

To this end the present invention suggests that the acupuncture needle comprises a coaxial light conducting element receiving an emitting biophoton radiation at it's puncturing end and cooperating with an interference element located within the grasping end of the needle.

According to a preferred embodiment of the invention the interference element comprises a prism with a 180° reflection.

Further details and characteristics of the invention are depicted in the drawing in combination with the following description.

The only drawing shows schematically an acupuncture needle according to the invention.

According to the invention the acupuncture needle 1 comprises a hollow needle body 2 in which light conduction element 3 of quartz glass is coaxially located. The puncturing end the light conducting element 3 extends over the end of the needle body 2 and is built as a puncturing point with a conical shape. With the extremity extending over the end of the needle body 2 the light conducting element 3 receives biophoton radiation and transmits it to a prism 4 with a 180° reflection. This prism 4 is fixed in a retention device 5 removably or fixedly located in a recess 6 at the grasping end of the needle body and holding it in contact with the proximal end of the light conducting element 3. This prism 4 is, e.g., a half cube of quartz glass which is in contact with the light conducting element 3 through its base face and which reflects the incoming biophoton radiation two times. By this arrangement the radiation coming from one roof face meets the radiation coming from the other roof face and is brought to interfere with each other and to be emitted again through the light conducting element 3 into the body tissue. Biophoton radiation is ideally adequate for this phenomenon due to its high coherence and low but stable intensity.

The reception and emission of biophoton radiation in or in the area of the puncture point has a particular therapeutical effect as these acupuncture areas present favorable properties with regard to electrical conductivity and biophoton radiation. With the acupuncture needle according to invention it is also possible to receive biophoton radiation from a minimal surface and to restitute it to the tissue after interference with counter current biophoton radiation.

By no means the embodiment of the invention shown in the drawing is restricted to the proportions and/or dimensions shown. They only have been chosen to illustrate the object of the invention. So, the puncturing end of the needle according to the invention may also present wedged end—such as an injection needle—so that the surface to receive and to emit biophoton radiation has ellipsoidal shape and thus is larger than a circular shape. Also the prism 4 may present any other adequate shape of an inverting prism.

What I claim is:

1. Acupuncture needle having a grasping and a puncturing end comprising a hollow needle body (2) presenting a coaxially located light conducting element (3) of quartz glass therein and having a reception and emission surface at the distal end of the needle, characterised by a prism (4) with a 180° reflection located within the proximal grasping end of the needle and in light conducting contact with the light conduction element (3).

2. Acupuncture needle according to claim 1 wherein the reception and emission distal end for the biophoton radiation of the needle (1) presents a conical shape.

3. Acupuncture needle according to claim 1 wherein the reception and emission distal end for the biophoton radiation of the needle (1) presents a tapered shape with an elliptical cross section.

4. Acupuncture needle according to claims 1 to 3 wherein the prism (4) within the proximal grasping end of the needle (1) is a half cube the base face of which is in light conducting contact with the light conducting element (3).
